(19) United States
(12) Patent Application Publication
(10) Pub. No.: US 2010/0305663 A1
(43) Pub. Date: Dec. 2, 2010

(54) IMPLANTABLE MEDICAL DEVICE SYSTEM HAVING SHORT RANGE COMMUNICATION LINK BETWEEN AN EXTERNAL CONTROLLER AND AN EXTERNAL CHARGER

(75) Inventor: DANIEL AGHASSIAN, GLENDALE, CA (US)

Correspondence Address: Wong, Cabello, Lutsch, Rutherford & Brucculeri LLP, 20333 SH 249, Suite 600, Houston, TX, 77070, US

(73) Assignee: BOSTON SCIENTIFIC NEUROMODULATION CORPORATION, VALENCIA, CA (US)

(21) Appl. No.: 12/476,523

(22) Filed: Jun. 2, 2009

(51) Int. Cl. A61N 1/378

(52) U.S. Cl. 607/61

(57) ABSTRACT

Disclosed is an improved system for providing charging information during the powering of a medical implantable device by an external changer. In the disclosed system, relevant charging information originates in the external charger, or is transmitted to the external charger from the implant during charging. The charging information is transferred from the external charger to an external controller using a short range communication link that is not orientation dependent (i.e., omni-directional), such as one employing a Bluetooth™ or Zibgee™ protocol for example. Once received, the external controller can convey the charging information to the patient or clinician, such as by displaying the charging information on the graphical user interface of the external controller. Additionally, the short range communication link between the external controller and the external charger allows the external charger to be controlled by the external controller, which adds system flexibility and convenience.

### FIELD OF THE INVENTION

The present invention relates to an improved implantable medical device system having a communication link between an external controller and an external charger.

### BACKGROUND

Implantable stimulation devices are devices that generate and deliver electrical stimuli to nerves and tissues for the therapy of various biological disorders, such as pacemakers to treat cardiac arrhythmia, defibrillators to treat cardiac fibrillation, cochlear stimulators to treat deafness, retinal stimulators to treat blindness, muscle stimulators to produce coordinated limb movement, spinal cord stimulators to treat chronic pain, cortical and deep brain stimulators to treat motor and psychological disorders, and other neural stimulators to treat urinary incontinence, sleep apnea, shoulder sublaxation, etc. The description that follows will generally focus on the use of the invention within a Spinal Cord Stimulation (SCS) system, such as that disclosed in U.S. Pat. No. 6,516,227. However, the present invention may find applicability in any implantable medical device system. For example, the disclosed invention can also be used with a Bion™ implantable stimulator, such as is shown in U.S. Patent Publication 2007/0097719, filed Nov. 3, 2005, or with other implantable medical devices.

As shown in FIGS. 1A and 1B, a SCS system typically includes an Implantable Pulse Generator (IPG) 100, which includes a biocompatible device case 30 formed of titanium for example. The case 30 typically holds the circuitry and battery 26 necessary for the IPG to function, although IPGs can also be powered via external RF energy and without a battery. The IPG 100 is coupled to electrodes 106 via one or more electrode leads (two such leads 102 and 104 are shown), such that the electrodes 106 form an electrode array 110. The electrodes 106 are carried on a flexible body 108, which also houses the individual signal wires 112 and 114 coupled to each electrode. In the illustrated embodiment, there are eight electrodes on lead 102, labeled E₁-E₈, and eight electrodes on lead 104, labeled E₉-E₁₆, although the number of leads and electrodes is application specific and therefore can vary. The leads 102 and 104 couple to the IPG 100 using lead connectors 38a and 38b, which are fixed in a header material 36, which can comprise an epoxy for example. In a SCS application, electrode leads 102 and 104 are typically implanted on the right and left side of the dura within the patient's spinal cord. These leads 102 and 104 are then tunneled through the patient's flesh to a distant location, such as the buttocks, wherein the IPG 100 is implanted.

As shown in cross section in FIG. 3, the IPG 100 typically includes an electronic substrate assembly 14 including a printed circuit board (PCB) 16, along with various electronic components 20, such as a microcontroller, integrated circuits, and capacitors mounted to the PCB 16. Two coils are generally present in the IPG 100: a telemetry coil 13 used to transmit/receive data to/from an external controller 12; and a charging coil 18 for charging or recharging the IPG's battery 26 using an external charger 50. The telemetry coil 13 can be mounted within the header 36 of the IPG 100 as shown.

FIG. 2 shows plan views of the external controller 12 and the external charger 50, and FIG. 3 shows these external devices in cross section and in relation to the IPG 100 with which they communicate. The external controller 12, such as a hand-held programmer or a clinician's programmer, is used to send data to and receive data from the IPG 100. For example, the external controller 12 can send programming data such as therapy settings to the IPG 100 to dictate the therapy the IPG 100 will provide to the patient. Also, the external controller 12 can act as a receiver of data from the IPG 100, such as various data reporting on the IPG's status. As shown in FIG. 3, the external controller 12, like the IPG 100, also contains a PCB 70 on which electronic components 72 are placed to control operation of the external controller 12. The external controller 12 is powered by a battery 76, but could also be powered by plugging it into a wall outlet for example. A telemetry coil 73 is also present in the external controller 12, which coil will be discussed further below.

The external controller 12 typically comprises a graphical user interface 74 similar to that used for a portable computer, cell phone, or other hand held electronic device. The graphical user interface 74 typically comprises touchable buttons 80 and a display 82, which allows the patient or clinician to operate the external controller 12, to send programs to the IPG 100, and to review any relevant status information that has been reported from the IPG 100 during its therapeutic operation.

Wireless data transfer between the IPG 100 and the external controller 12 preferably takes place via inductive coupling, although a higher radiofrequency link could also be used. To implement indicative coupling functionality, both the IPG 100 and the external controller 12 have coils 13 and 73 respectively. Either coil can act as the transmitter or the receiver, thus allowing for two-way communication between the two devices. Referring to FIG. 4, when data is to be sent from the external controller 12 to the IPG 100 (170), coil 73 is energized with alternating current (AC), which generates a magnetic field, which in turn induces a voltage in the IPG's telemetry coil 13. The generated magnetic field is typically modulated (120) using a communication protocol, such as a Frequency Shift Keying (FSK) protocol, which is well known in the art. The induced voltage in coil 13 can then be demodulated (125) at the IPG 100 back into the telemetered data signals. Data telemetry in the opposite direction (172) from IPG 100 to external controller 12 occurs similarly. This means of communicating by inductive coupling is transcutaneous, meaning it can occur through the patient's tissue 25.

The external charger 50 is used to charge (or recharge) the IPG's battery 26. Specifically, and similarly to the external controller 12, the external charger 50 contains a coil 88 which is energized via charging circuit 122 with a non-modulated AC current to create a magnetic charging field (174). This magnetic field induces a current in the charging coil 18 within the IPG 100, which current is rectified (132) to DC levels, and used to recharge the battery 26, perhaps via a charging and battery protection circuit 134 as shown. Again, inductive coupling of power in this manner occurs transcutaneously.

The IPG 100 can also communicate data back (176) to the external charger 50 using modulation circuitry 126. Modulation circuitry 126 receives data to be transmitted back to the external charger 50 from the IPG's microcontroller 150, and then uses that data to modulate the impedance of the charging coil 18. In the illustration shown, impedance is modulated via control of a load transistor 130, with the transistor's on-resistance providing the necessary modulation. This change in impedance is reflected back to coil 88 in the external charger 50, which interprets the reflection at demodulation circuitry 123 to recover the transmitted data. This means of transmitting data from the IPG 100 to the external charger 50 is known as Load Shift Keying (LSK), and is useful to communicate data relevant during charging of the battery 26 in the IPG 100, such as the capacity of the battery, whether charging is complete and the external charger can cease, and other pertinent charging variables. However, because LSK works on a principle of reflection, such data can only be communicated from the IPG 100 to the external charger 50 during periods in which the external charger is active and is producing a magnetic charging field (174).

As shown in FIG. 3, the external charger 50 generally comprises at least one printed circuit board 90, electronic components 92 which control operation of the external charger 50, and a battery 96 for providing operational power for the charger 50 and for the production of the magnetic charging field. Like the external controller 12, the external charger 50 has a user interface 94 to allow the patient or clinician to operate the charger 50. The user interface 94 typically comprises an on/off switch 95 which activates the production of the magnetic charging field; an LED 97 to indicate the status of the on/off switch 95; and a speaker 98 for emitting a “beep” at various times. For example, the speaker 98 can beep if the charger 50 detects that its coil 88 is not in good alignment with the charging coil 18 in the IPG 100. In a SCS application in which the IPG 100 is implanted in the patient's buttocks, the external charger 50 is generally held against the patient's skin or clothes and in good alignment with the IPG 100 by a belt or an adhesive patch, which allows the patient some mobility while charging. In short, the external charger 50 is positioned behind the patient.

As one might appreciate from the foregoing description, the user interface 94 of the external charger 50 is generally simpler than the graphical user interface 74 of the external controller 12. Such user interface simplicity is understandable for at least two reasons. First is the relative simplicity of the charging function the external charger 50 provides. Second, a complicated user interface, especially one having visual aspect, may not be warranted because the external charger 50 may not be visible to the patient when it is used. For example, in a SCS application, the external charger 50 would generally be behind the patient to align properly with the IPG 100 implanted in the buttocks as just discussed. The external charger 50 would not be visible, and thus providing the user interface 94 of the external charger 50 with a display or other visual indicator would be of questionable benefit. Additionally, the external charger 50 may be covered by clothing, again reducing the utility of any visual aspect to the user interface.

Although the simplicity of the user interface 94 of the external charger 50 is understandable, the inventor still finds such simplicity regrettable. Even if operation of the external charger 50 is relatively simple, the fact remains that several pieces of information relevant to the charging process might be of concern to the patient, which charging information is impractical or impossible to present by audible means such as through speaker 98.

For example, it may be desired for the user to have some information concerning the alignment between the external charger 50 and the IPG 100. Or, the user may wish to know the status of the implant battery, i.e., to what level it is charged, and how much longer charging might take. The user may also wish to know when the implant battery is fully charged, such that charging can cease. In another example, it may be of benefit for the user to know the temperature of the external charger 50. Typically, this temperature is monitored by a thermocouple 101 in the external charger 50. If the temperature exceeds a temperature which might be harmful to the patient (e.g., 41° C.), then the logic in the external charger 50 takes automatic steps to remedy this issue, such as by temporarily suspending production of the magnetic charging field. Regardless, despite the importance of the temperature of the external charger 50, the user interface 94 does not present such information to the user.

Likewise, it may be desired for the user to know the status of the external charger's battery 96; the relative degree or direction of misalignment between the external charger 50 and the IPG 100; when charging of the IPG's battery 26 has completed, etc. But again, it is impractical to present such information to the user by audible means. If used in a loud environment, if the external charger 50 is audibly obstructed, or if the patient is hard of hearing, audible indicators are that much less effective.

Given these shortcomings, the art of implantable medical devices would benefit from an improved means for providing relevant charging information to a patient, and this disclosure presents solutions.

### BRIEF DESCRIPTION OF THE DRAWINGS

FIGS. 1A and 1B show an implantable pulse generator (IPG), and the manner in which an electrode array is coupled to the IPG in accordance with the prior art.

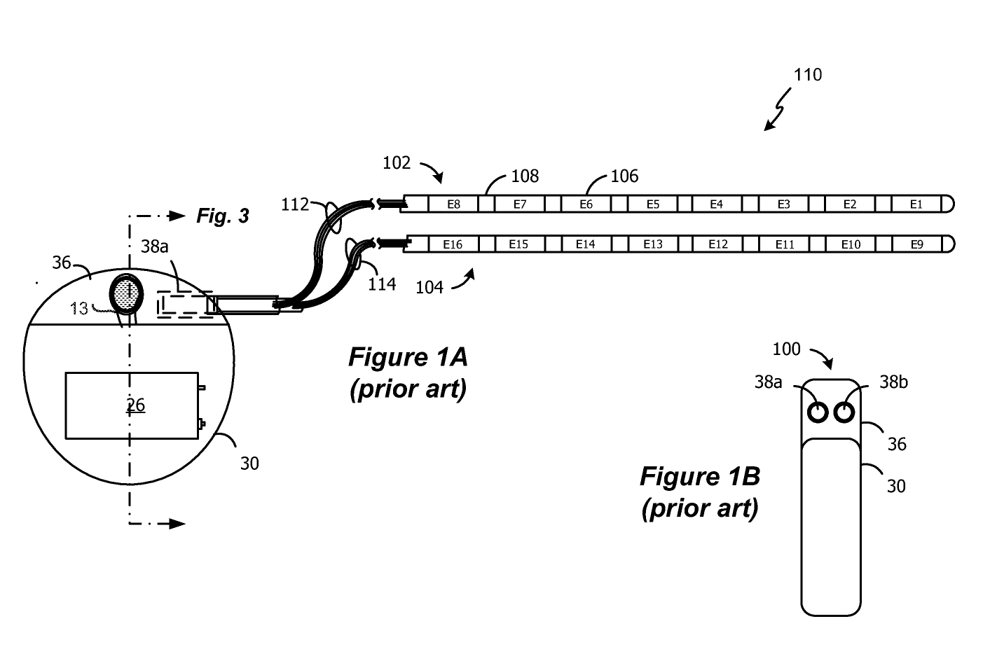

FIG. 2 shows plan views of an external controller and an external charger which communicate with an IPG in accordance with the prior art.

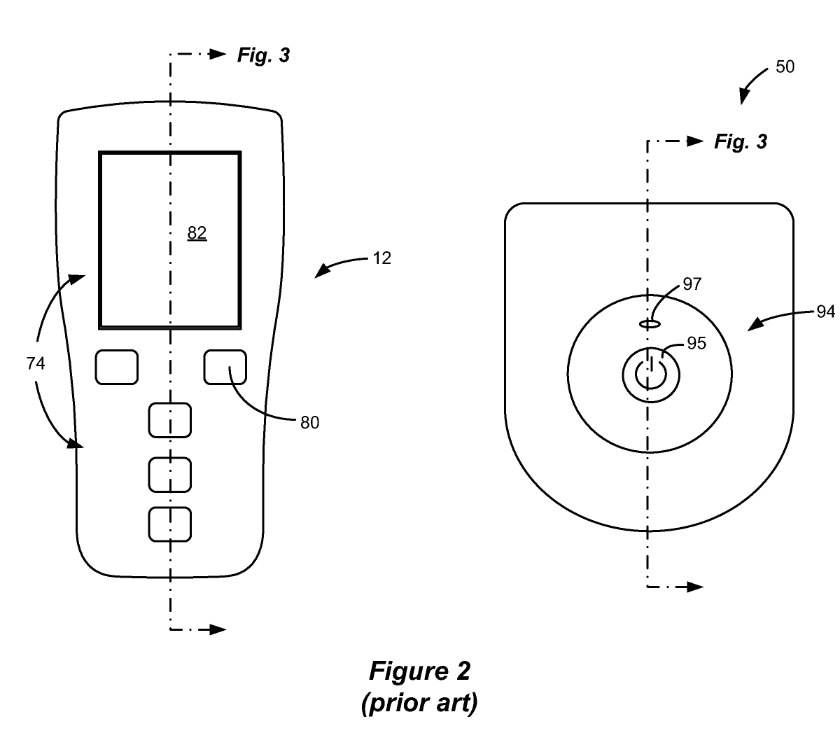

FIG. 3 shows cross sectional views of the external controller, the external charger and the IPG of FIGS. 1 and 2, and shows the communicative relations between these devices.

FIG. 4 shows the communication circuitry present in the external controller, the external charger, and the IPG in accordance with the prior art.

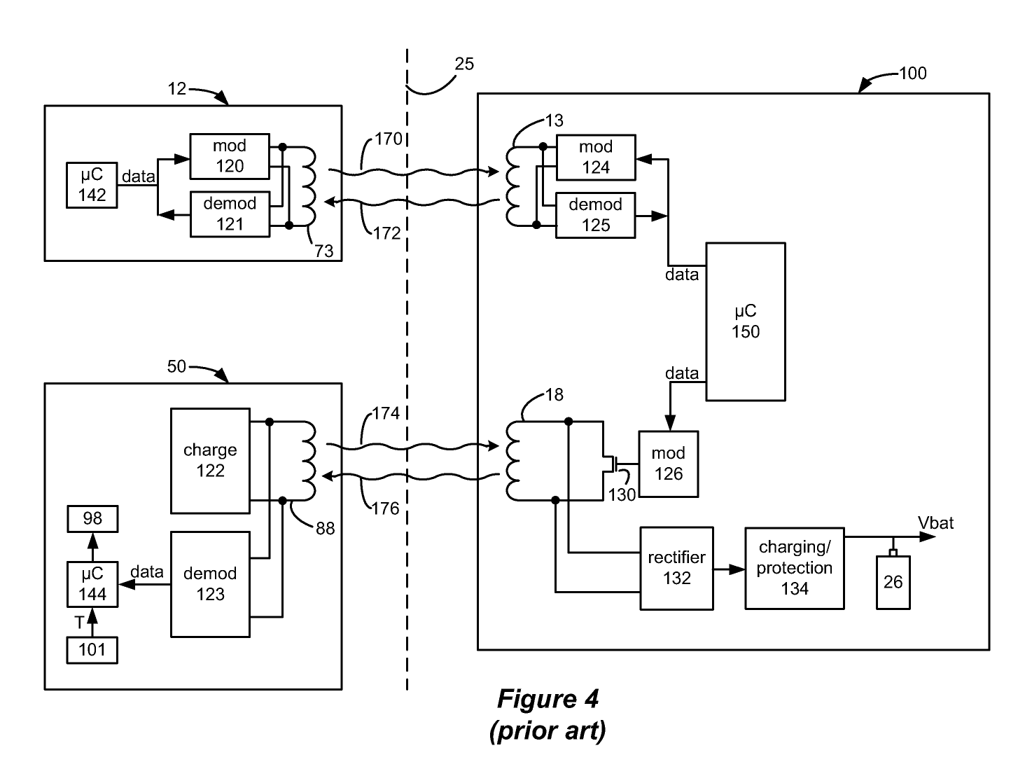

FIG. 5 shows an improved system for providing charging status information in accordance with an embodiment, in which the external charger and external controller can communicate via a RF communication link.

FIG. 6 shows the communication circuitry present in the external controller, the external charger, and the IPG in the improved system of FIG. 5.

FIG. 7 shows the graphical user interface of the external controller, and how that interface can display charging information.

FIG. 8 shows an additional computer useable in the improved system of FIG. 5 which can communicate with both the external controller and the external charger via RF communication links.

### DETAILED DESCRIPTION

The description that follows relates to use of the invention within a spinal cord stimulation (SCS) system. However, it is to be understood that the invention is not so limited. Rather, the invention may be used with any type of implantable medical device system. For example, the present invention may be used in a system employing an implantable sensor, an implantable pump, a pacemaker, a defibrillator, a cochlear stimulator, a retinal stimulator, a stimulator configured to produce coordinated limb movement, a cortical and deep brain stimulator, or in any other neural stimulator system configured to treat any of a variety of conditions.

Disclosed is an improved system for providing charging information during the powering of a medical implantable device by an external changer. In the disclosed system, relevant charging information originates in the external charger, or is transmitted to the external charger from the implant during charging. The charging information is then transferred from the external charger to an external controller using a short range communication link that is not orientation dependent (i.e., omni-directional), such as one employing a Bluetooth™ or Zibgee™ protocol for example. Once received, the external controller can convey the charging information to the patient or clinician, such as by displaying the charging information on the graphical user interface of the external controller. Because the external controller, unlike a typical external charger, has a graphical user interface and can be positioned in a convenient location in front of the patient, the charging information is easily and comprehensibly conveyed to the patient. Additionally, the short range communication link between the external controller and the external charger allows the external charger to be controlled by the external controller, which adds system flexibility and convenience.

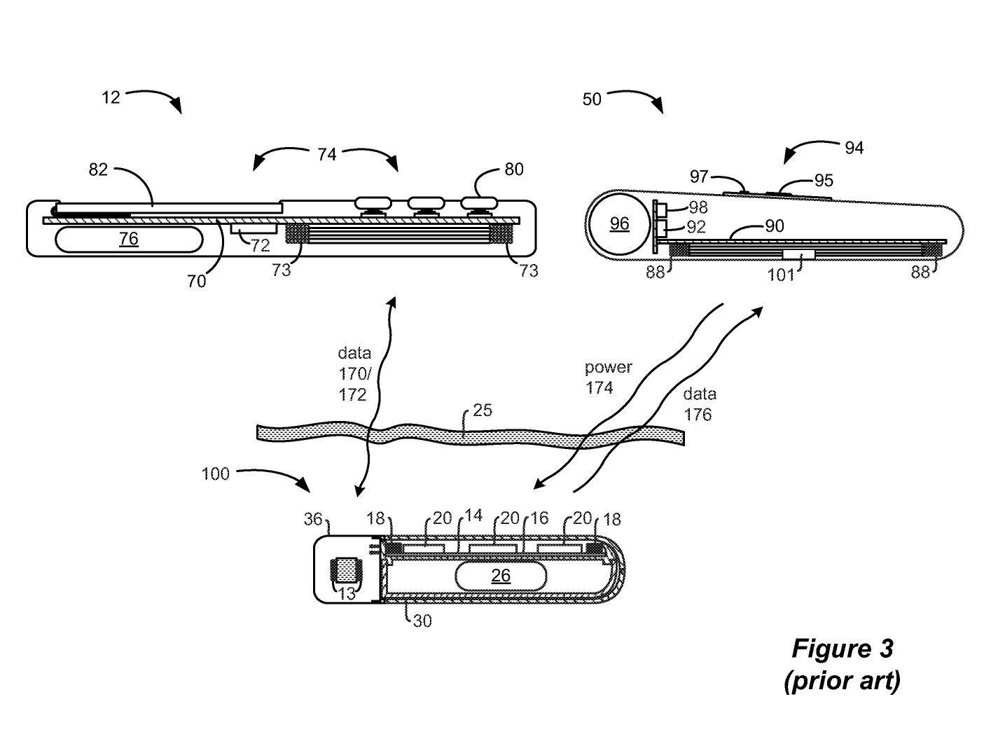

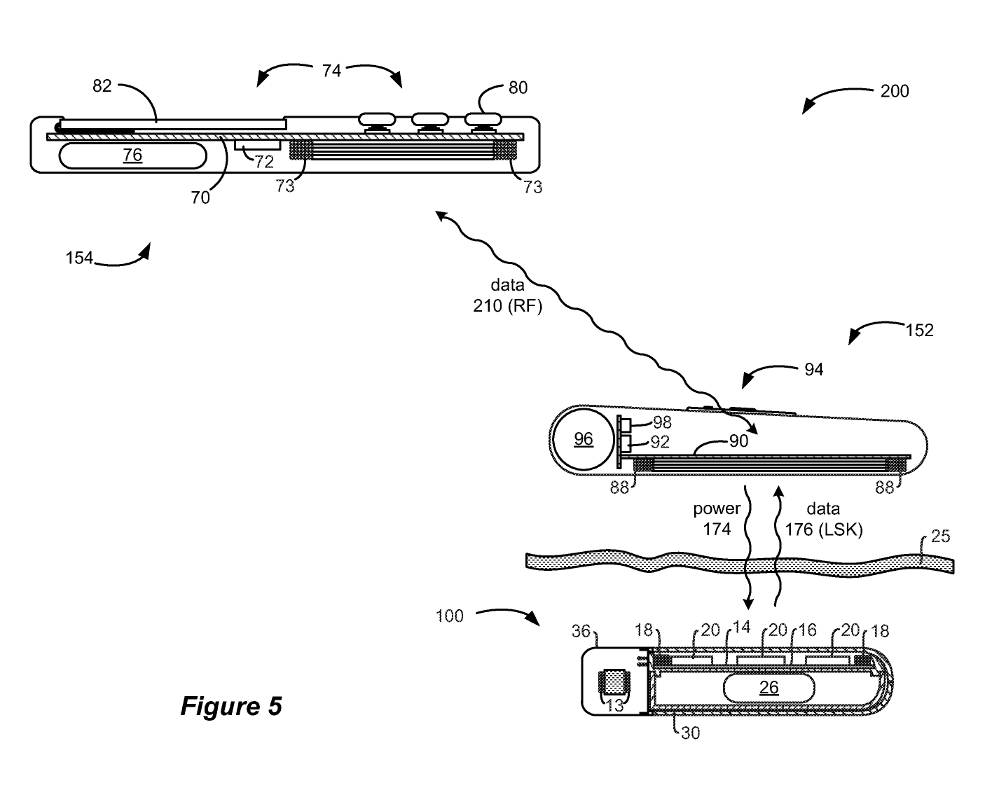

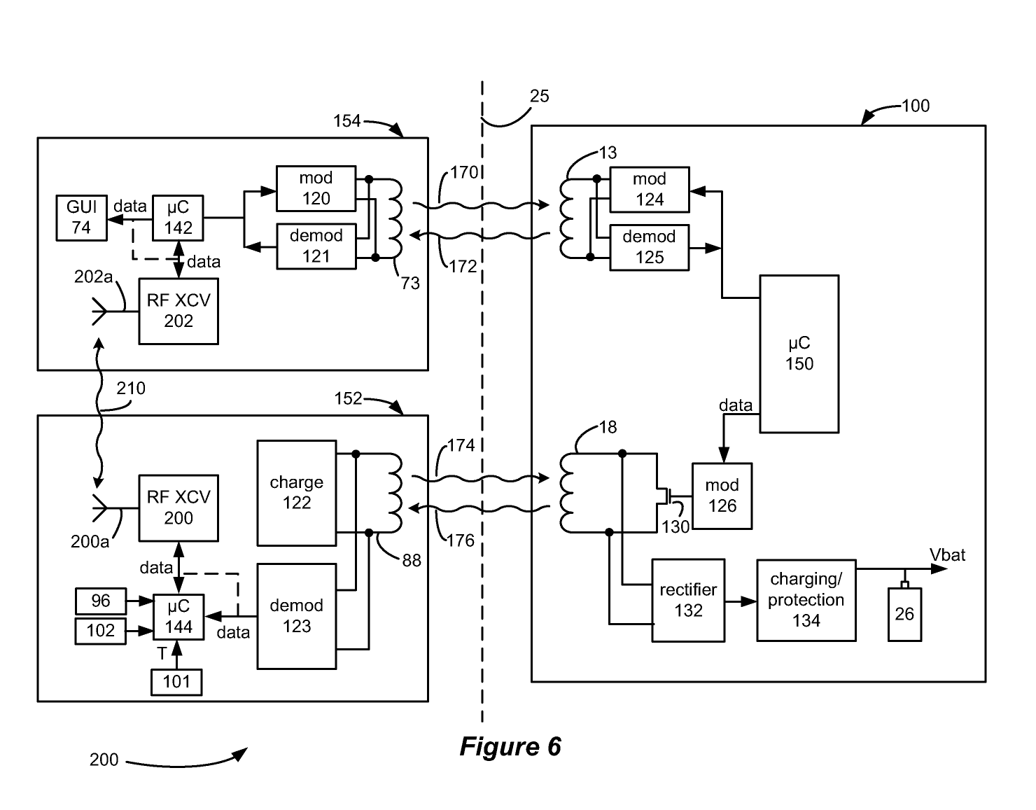

FIGS. 5 and 6 disclose an embodiment of the just-mentioned improved system 200 for providing charging information, which comprises an IPG 100, an improved external charger 152, and an improved external controller 154. Unlike previously-known charging approaches, the improved system 200 uses an external controller 154 as part of the system, and in particular as the part of the system which ultimately conveys the charging information to the patient. In previously-known systems, such as that illustrated in FIG. 3, external controllers were discrete from the external chargers, and due to the division of functions between these two devices, were not involved during the charging process.

In system 200, the external charger 152 and IPG 100 can communicate in the same manner as they did in previous systems: coil 88 is used to create a magnetic charging field 174 which is received and rectified at the IPG 100 and used to charging the IPG's battery 26 in the manner preciously described; data 176, including relevant charging information, can be reported back to the external charger 152 by LSK for example, etc. The external controller 154 can also communicate with the IPG 100 as it did in the prior art via data links 170 and 172, such as by sending therapy settings to the IPG during non-charging periods. The external charger 152 has a user interface 94, and external controller 154 has a graphical user interface 74 similar to those described earlier, and each has their own housings.

However, unlike in previous systems, charging information, i.e., information generated during charging and relevant to the charging process, is sent to the external controller 154 to take advantage of the controller's improved graphical user interface 74, and possibly also its data processing power. As shown in FIGS. 5 and 6, this can occur by establishing a short range RF communications link 210 between the external controller 154 and the external charger 152. Such link may be enabled by RF transceivers (XCVs) 200 and 202 and their associated antennas 200a and 202a in the charger 152 and controller 154 respectively. Link 210 preferably comprises a Bluetooth™ compliant link, but any other suitable communications protocol, e.g., Zigbee™, WiFi, CMDA, TDMA, etc., could be used as well. Bluetooth™ is preferred as a standard, low power, low cost option which still provides a reasonable communication range (distance) between the external charger 152 and the external controller 154. The RF communication link 210 is preferably bi-directional, although many of the benefits to the use of this link as disclosed will focus on sending of data from the external charger 152 to the external controller 154.

FIG. 6 illustrates details of the communication circuitry used to send charging information to the external controller 154. Charging information reported to the external controller 154 can comprise data originating from either the IPG 100 or the external charger 152 itself. For example, the capacity of the IPG's battery 26 (Vbat) may be relevant for a user to review during charging and such data would ultimately originate at the IPG 100. As such it would be reported to the external charger 152 via link 176. For simplicity, link 176 is preferably implemented as an FSK link, and so shares the hardware (e.g., coils 88 and 18) used to provide power 174 to the IPG 100. However, link 176 could also be implemented as a separate link not having any connection to the power link 174, although such separate link would require additional hardware. Still, link 176 may be implemented by any means suitable to transmitting data from the IPG 100 to the external charger 152 during charging.

Once such charging information (e.g., Vbat) arrives at the external charger 152, it is demodulated (123) as necessary, and readied for transmission to the external controller 154 via RF link 210. Readying the data for transmission can comprise processing of such data using the external charger's microcontroller 144, or the microcontroller 144 can be bypassed as shown in dotted lines in FIG. 6. For example, if Vbat as demodulated (123) is already is a digital form understandable by the external controller 154, it can be simply sent directly from the demodulator 123 to the RF transceiver 200. However, in some instances, data received from the IPG 100 may not be in the correct digital form, or it may otherwise be desirable to process the received data at the external charger 152 prior to transmission to the external controller 154. In this case, microcontroller 144 is programmed to perform an algorithm on the received data. In a simple example, it may be preferable to average the battery voltage (Vbat) data over some period of time before such data is sent to the external controller 154. More significant processing of data reported from the IPG 100 may occur at the external charger 152's microcontroller 144 prior to reporting the same to the external controller 154.

As mentioned above, some relevant charging information originates at the external charger 152 itself. Some examples of such information include alignment information, which may come from alignment circuitry 102 in the external charger 152. Such alignment information informs concerning the physical relationship between the alignment of the external charger 152 relative to the IPG 100, including how the external charger 152 should be moved to try and improve the coupling between the two to make charging more efficient. Example alignment circuitry 102 is disclosed in a U.S. patent application entitled “An Improved External Charger for a Medical Implantable Device Using Field Sensing Coils to Improve Coupling,” Attorney Docket Number 585-0069US, filed [date]. Other charging information generated at the external charger could comprise the charger's temperature, T, as provided by thermocouple 101, and the capacity of the charger's battery 96. In any event, such charger-originated charging information, like the IPG-originated charging information, can be processed at the charger's microcontroller 144, or passed directly to the transceiver 200 essentially unmodified.

Regardless of whether the charging information originated in the IPG 100, the external charger 152, or both, that data is reported from the external charger 152 to the external charger 154 using RF communication link 210, as discussed above. Upon receipt at the transceiver 202 in the external controller 154, the received data is ultimately presented at the graphical user interface 74 of the external controller 154, although the data may be processed first using the controller's microcontroller 142. As was the case with the external charger 152, the microcontroller 142 in the external controller 154 can process the received data in accordance with any number of algorithms. Such algorithms may generally be designed to present the received changing information in an appropriate and clear way at the graphical user interface 74. For example, if the relevant data to be reviewed by the user is the external charger's temperature data, the microcontroller 142 may format the data to be displayed on the graphical user interface 74 as a graph so that changes in that data can be seen over time. Alternatively, or in conjunction with display of charging information at the graphical user interface, the charging information may also be conveyed to the patient using non-graphical aspects of the user interface 74, such as by sounds (beeps or voices), by lights, by vibration, or by other non-graphical means.

Processing at the microcontroller 142 may also involve analysis of the data. In another example, the algorithm operating at the microcontroller 142 may assess the reported external charger temperature data to make a determination whether the temperature is unsafe, or has been or is forecasted to be unsafe, with the result being an indication to the user of an unsafe condition. If a bi-directional communication link 210 is used, such analysis may also result in communication of control instructions back to the external charger 152. For example, if microcontroller 142 determines that the charger temperature T is too high, the microcontroller 142 in the external controller 154 can instruct the microcontroller 144 in the external charger to discontinue charging.

Even absent use of the external controller's user interface 74, the inclusion of a RF communications link 210 allows processing of charging information to be offloaded to the external controller 154. Processing of charging information at the external controller 154 instead of the external charger 152 may be preferable, in particular if the processing resources at the external controller 152 are relatively lacking.

Unlike communication via inductive coupling, such as occurs in links 170, 172, and 176, communication via RF communication link 210 is not dependent on any particular alignment between the external charger 152 and the external controller. Whereas the external charger 152 may need to be placed in an inaccessible location proximate to the IPG 100 during charging, such as behind the patient, the external controller 154 can be held in the user's hands where it can be easily seen. This alleviates problems discussed earlier affecting prior art charging systems: because the external controller 154 has a more detailed (graphical) user interface 74 when compared to the user interface 94 of the external charger 152, and because the external controller 154 can essentially be located anywhere during the charging process, important charging information is more easily conveyed to the user.

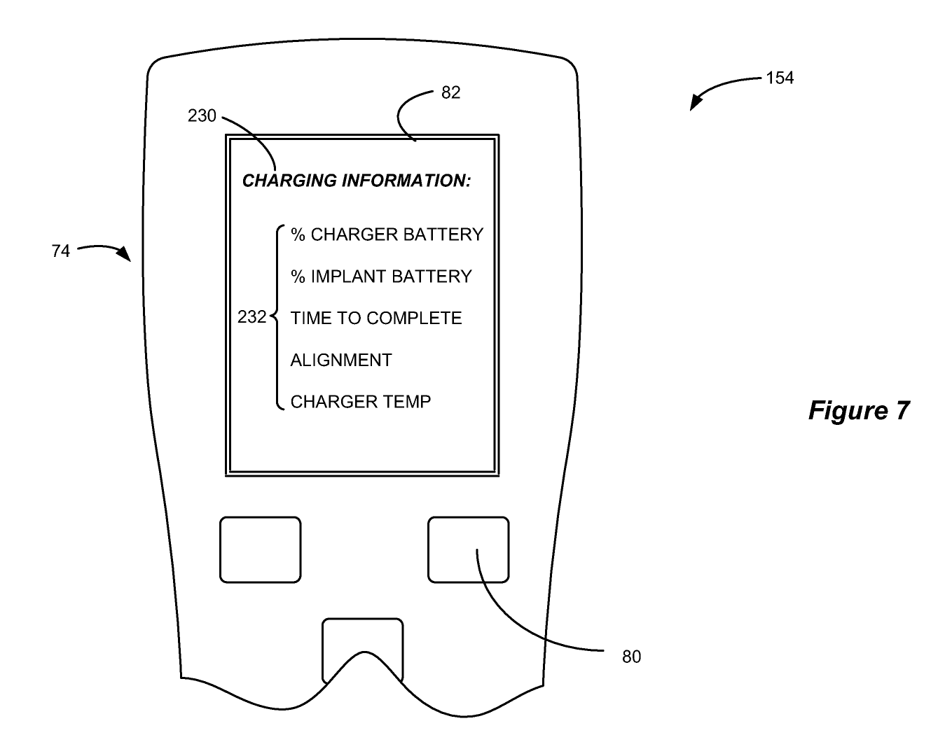

FIG. 7 shows the graphical user interface 74 of the external controller 154 as used to display various pieces of charging information, which information may or may not have been pre-processed as discussed earlier. As shown, the various pieces of charging information are displayed in a menu 232, from which the user may selected a desired entry using standard means. For example, as shown, the user can select to review: the capacity of both the implant and charger batteries, an estimated time to completion for charging, and alignment information, such as whether the user needs to move the external charger up or down, or left or right, relative to the implant, the charger temperature, etc. Such convenient means of conveying such charging information to the user was not possible using known charging system.

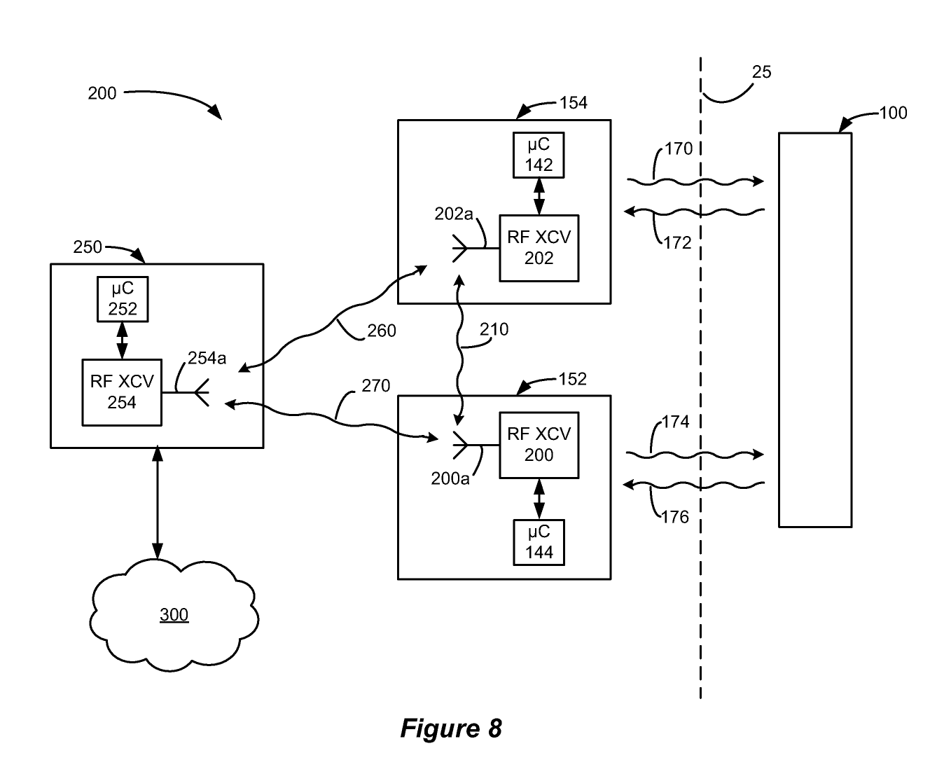

Because the illustrated embodiment relies on short range RF transceivers 202 and 200 to establish the communication link 210 between the external charger 152 and the external controller 154, those same transceivers can be put to additional advantageous uses. For example, as shown in FIG. 8, a third external device 250 is shown, complete with its own RF transceiver 254 and associated antenna 254a. External device 250 may comprises a generic computing device, such as a personal computer, a notebook computer, a PDA or PDA-like device, a cellular telephone, etc., but in a preferred application comprises a clinician's computer. Because the clinician's computer 250 contains the requisite protocol-compliant hardware, it may establish RF links 260 and 270 with the external controller 154 and the external charger 152, with such links being essentially identical to communication link 210 between the external controller 154 and the external charger 152.

With communication links 260 and 270 established, the clinician's computer 250 can communicate with the external controller 154 and the external charger 152. This is useful for many reasons, including reasons not relating to use of the external charger 152 to charge the IPG 100's battery 26. For example, links 260 and 270 may be used to download data from either of devices 154 or 152, to update or configure the operating software in those devices, to trouble-shoot those devices, etc. Because a clinician's computer 250 may be easily connected to another network, such as the internet 300, the communication capabilities of system 200 are further extendable to many useful ends. Additionally, the same charging information discussed earlier can be conveyed to the user via the user interface at the clinician's computer 250, either as sent directly from the external charger 152 via link 270, or via the external controller 154 as an intermediary via links 210 and 260.

Because external devices 250 such as a clinician's computer will have, or can easily be made to have, Bluetooth-compliant hardware, the choice of Bluetooth as the protocol for communication link 210 facilitates networking of the external charger 152 and external controller 154 with such devices 250. However, should system 200 comprise only the external charger 152 and the external controller 154, then another protocol such as Zibgee™, which is not as ubiquitous but which has advantages in the context of medical implantable device communications, might be preferred.

It should be noted that while the magnetic charging field (174) produced by the external charger 152 is generally used to charge a battery in the IPG 100, that same magnetic charging field may be used to provide in real time power to an IPG lacking a battery or other energy storage medium. The disclosed techniques are applicable to such battery-less implant applications.

Although the disclosed embodiments tout the benefits of providing a communication link 210 between the external charger 152 and the external controller 154, and note that the external controller 154 can enhance the operation of the external charger 152, it is preferred in system 200 that operation of the external charger 152 is not dependent on the external controller 154. In other words, should the external controller 154 not be present, should the communication link 210 be unreliable because of interference or other factors, or should the user simply decide not to use the external controller 154 during charging, the external charger 152 can act independently to charge the IPG's battery 26, and thus retains the full and independent functionality of external chargers traditionally found in the prior art.

Although particular embodiments of the present invention have been shown and described, it should be understood that the above discussion is not intended to limit the present invention to these embodiments. It will be obvious to those skilled in the art that various changes and modifications may be made without departing from the spirit and scope of the present invention. Thus, the present invention is intended to cover alternatives, modifications, and equivalents that may fall within the spirit and scope of the present invention as defined by the claims.

What is claimed is:

1. A system for providing power to an implantable medical device, comprising:
an external charger for providing power to the implantable medical device by producing a magnetic charging field; and
an external controller for sending therapy settings to the implantable medical device,
wherein charging information is transferred from the external charger to the external controller along a wireless communication link during providing power to the implantable medical device by the external charger.

2. The system of claim 1, wherein the external controller further comprises a user interface for conveying the transferred charging information to a user.

3. The system of claim 2, wherein the user interface is graphical.

4. The system of claim 3, wherein the external charger further comprises a non-graphical user interface.

5. The system of claim 1, wherein the wireless communication link is an RF wireless communication link that is not dependent on alignment between the external charger and the external controller.

6. The system of claim 5, wherein the wireless communication link comprises a Bluetooth link or a Zigbee link.

7. The system of claim 1, wherein the charging information originates in the implantable medical device, the external charger, or both.

8. The system of claim 1, wherein the wireless communication link is bi-directional.

9. The system of claim 1, wherein the external controller and the external charger each have their own housings.

10. The system of claim 1, wherein the wireless communication link comprises a protocol, and wherein the system further comprises an external device capable of wirelessly communicating with the external controller and the external charger via the protocol.

11. A method for providing power to an implantable medical device, comprising:
activating an external charger to provide power to the implantable medical device;
receiving charging information at the external charger during providing power to the implantable medical device;
wirelessly transmitting the charging information to an external controller along a communication link during providing power to the implantable medical device.

12. The method of claim 11, further comprising conveying the charging information to a user via a user interface on the external controller.

13. The method of claim 12, wherein the user interface is graphical.

14. The method of claim 13, wherein the external charger further comprises a non-graphical user interface.

15. The method of claim 11, wherein the communication link is an RF wireless communication link that is not dependent on alignment between the external charger and the external controller.

16. The method of claim 15, wherein the communication link comprises a Bluetooth link or a Zigbee link.

17. The method of claim 11, wherein the charging information originates in the implantable medical device, the external charger, or both.

18. The method of claim 11, further comprising transmitting therapy settings from the external controller to the implantable medical device.

19. The method of claim 11, wherein the charging information comprises information indicative of the temperature of the external charger.

20. The method of claim 11, wherein the charging information comprises information indicative of the capacity of the battery in the external charger, the capacity of the battery in the implantable medical device, or both.

21. The method of claim 11, wherein the charging information comprises information regarding the alignment between the external charger and the implantable medical device.

22. The method of claim 11, further comprising sending via the communication link at least one control instruction back from the external controller to the external charger in response receipt of the charging information at the external controller.

23. The method of claim 22, wherein the at least one control instruction comprises an instruction to cease activating the external charger to provide power to the implantable medical device.

24. The method of claim 11, wherein power is provided to the implantable medical device to charge a battery in the implantable medical device.
